(19) United States
(12) Patent Application Publication
(10) Pub. No.: US 2021/0343371 A1
(43) Pub. Date: Nov. 4, 2021

(54) HIGH-THROUGHPUT INFECTIOUS DISEASE DIAGNOSIS PLATFORM

(71) Applicant: Intellect Galore, Inc., Los Angeles, CA (US)

(72) Inventors: Eung Joon Jo, West New York, NJ (US); Chung-Kon Ko, Seongnam-si (KR)

(73) Assignee: Intellect Galore, Inc., Los Angeles, CA (US)

(21) Appl. No.: 17/245,006

(22) Filed: Apr. 30, 2021

Related U.S. Application Data
(60) Provisional application No. 63/018,530, filed on May 1, 2020.

(51) Int. Cl. G16B 30/00; G16B 50/00; G16B 40/00; G16H 10/40; G06N 3/12

(52) U.S. Cl. CPC G16B 30/00; G16B 50/00; G06N 3/123; G16H 10/40; G16B 40/00

(57) ABSTRACT

A sample (e.g. saliva and/or blood) may be takes from a test subject (e.g. a human patient). The sample may be processes to extract a testable substance. The testable substance may be processed by a mass spectrometer in order to generate a mass spectrometry profile of the sample. If the sample includes an infectious disease, then the infectious disease may be reflected in the mass spectrometry profile. The mass spectrometry profile may be compared to a database of mass spectrometry profiles from samples of test subjects whom have already be diagnoses with a particular infectious disease. If the mass spectrometry profile of the test subject being diagnosed is substantially similar to one or more mass spectrometry profiles in the predetermined database, then a diagnosis of an infection disease may be made in an accurate, efficient, and time sensitive manner.

The present application claims priority to U.S. Provisional Patent Application No. 63/018,530 filed on May 1, 2020, which is hereby incorporated by reference in its entirety.

### BACKGROUND

The propagation of infectious diseases is a serious contemporary problem. For example, the COVID-19 pandemic that originated in 2019 has caused worldwide disruption and a significant amount of human death. Likewise, previous epidemics (e.g. SARS originating in 2003 or MERS originating in 2012) also posed a worldwide challenges. In efforts to control epidemics, pandemics, and/or other spreading of infectious diseases, diagnosis of the infectious disease in an accurate, efficient, and diligent manner are very important. For example, a chemical based testing method of polymerase chain reaction (i.e. PCR test) is used, but there are complications with PCR tests based on inaccuracies, inefficiencies, and the relatively long period of time to receive test results. Although there are rapid PCR tests, these rapid PCR tests tend to be highly inaccurate. Accordingly, there is a need for more accurate, efficient, and time sensitive manner to diagnose infectious diseases in patients.

### SUMMARY

In embodiments, a method using mass spectrometry and/or an apparatus using mass spectrometry analysis may be utilized to diagnose infectious diseases. A sample (e.g. saliva and/or blood) may be takes from a test subject (e.g. a human patient). The sample may be processes to extract a testable substance. The testable substance may be processed by a mass spectrometer in order to generate a mass spectrometry profile of the sample. If the sample includes an infectious disease, then the infectious disease may be reflected in the mass spectrometry profile. The mass spectrometry profile may be compared to a database of mass spectrometry profiles from samples of test subjects whom have already be diagnoses with a particular infectious disease. If the mass spectrometry profile of the test subject being diagnosed is substantially similar to one or more mass spectrometry profiles in the predetermined database, then a diagnosis of an infection disease may be made in an accurate, efficient, and time sensitive manner.

### DRAWINGS

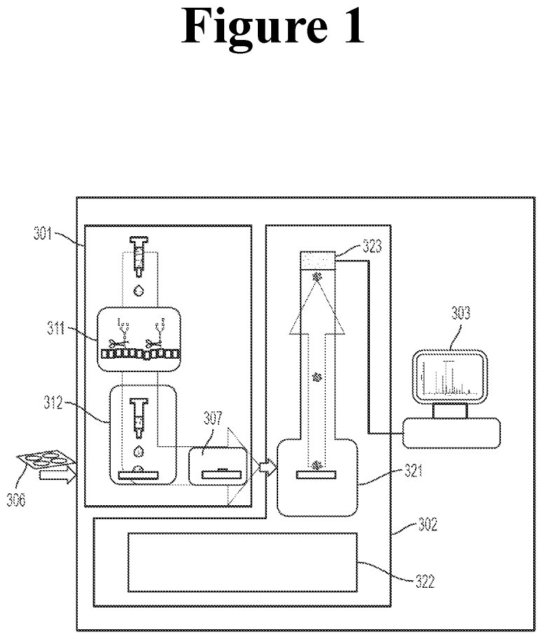

Example FIG. 1 is a system diagram of a mass spectrometry unit, in accordance with embodiments.

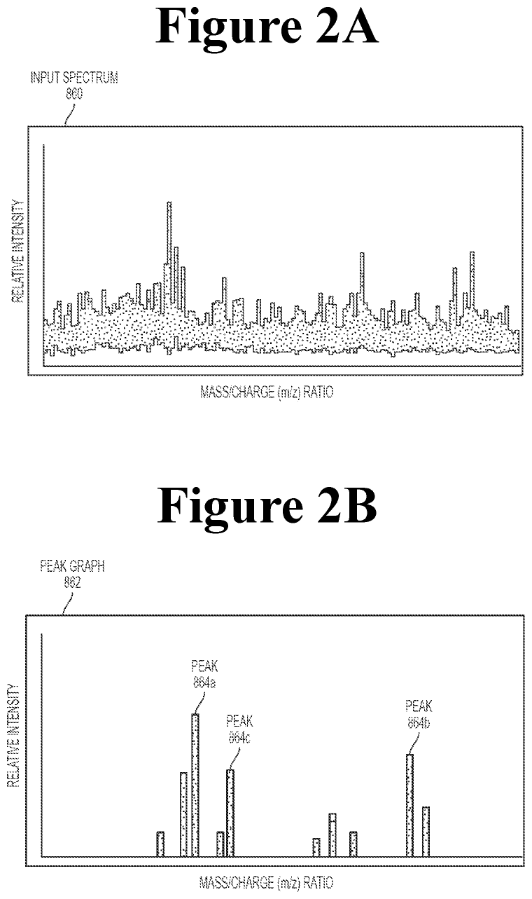

Example FIGS. 2A and 2B show and example mass spectrometry input spectrum and corresponding graph of peaks of input spectrum, in accordance with embodiments.

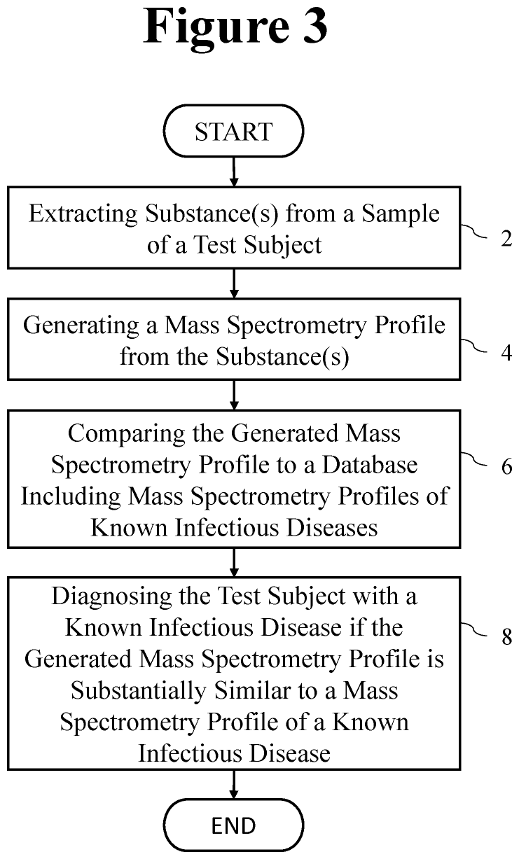

Example FIG. 3 illustrates a process of diagnosing infectious diseases, in accordance with embodiments.

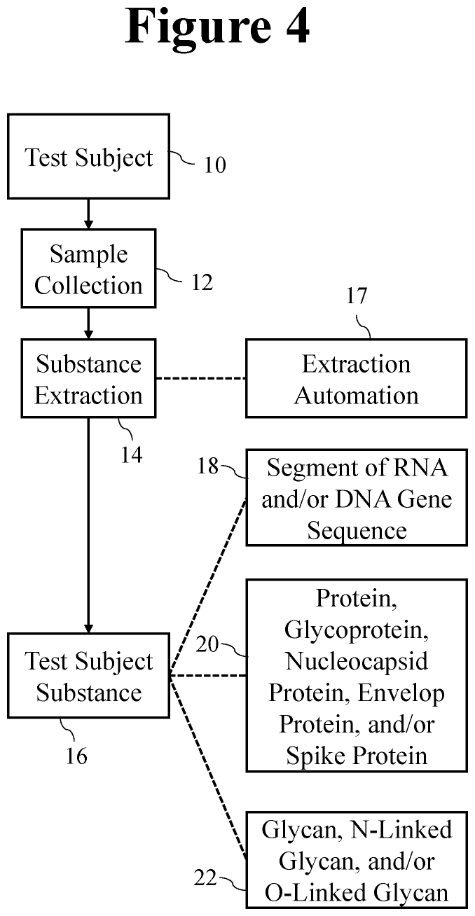

Example FIG. 4 illustrates substance extraction from a test subject, in accordance with embodiments.

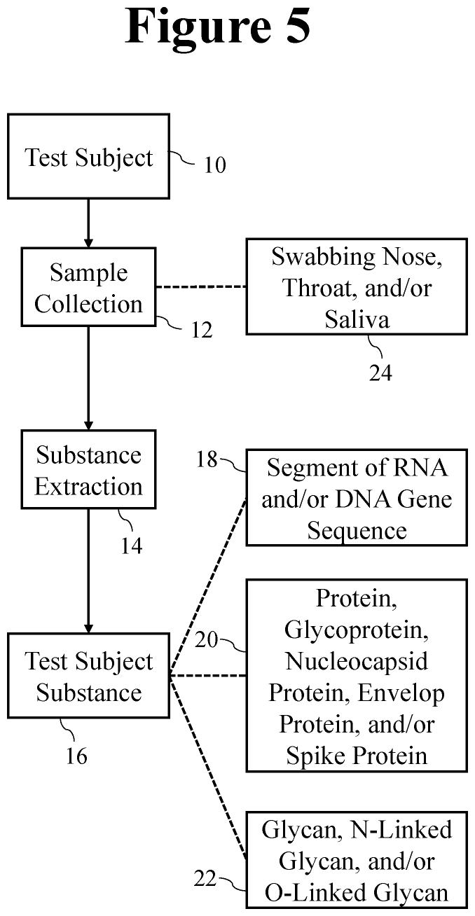

Example FIG. 5 illustrates swabbing extraction from nose, throat, and/or saliva from a test subject, in accordance with embodiments.

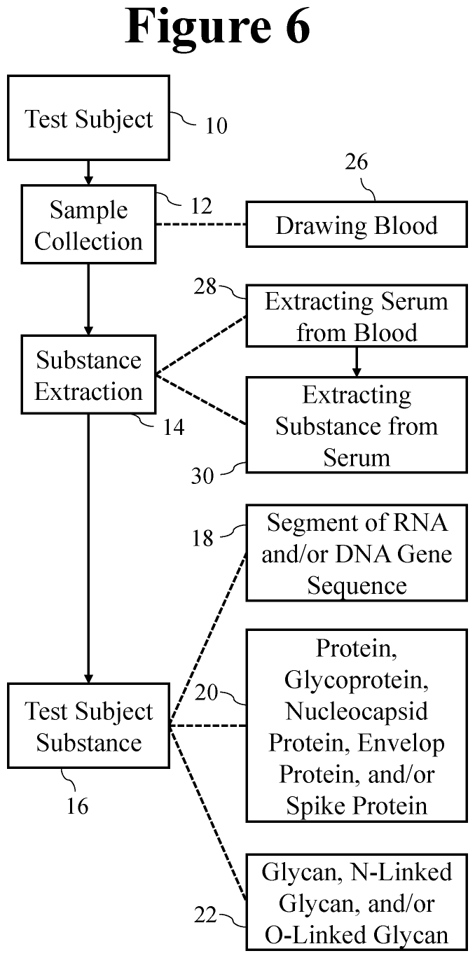

Example FIG. 6 illustrates blood extraction from a test subject, in accordance with embodiments.

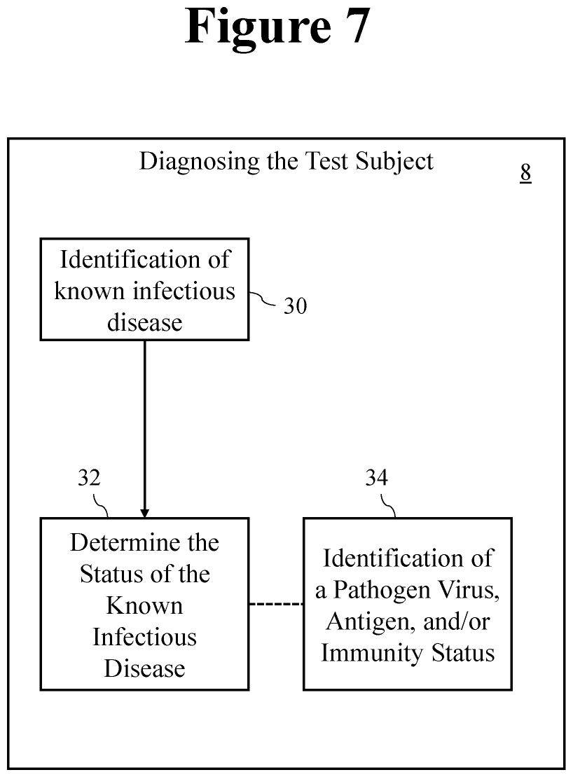

Example FIG. 7 illustrates diagnosis of a test subject, in accordance with embodiments.

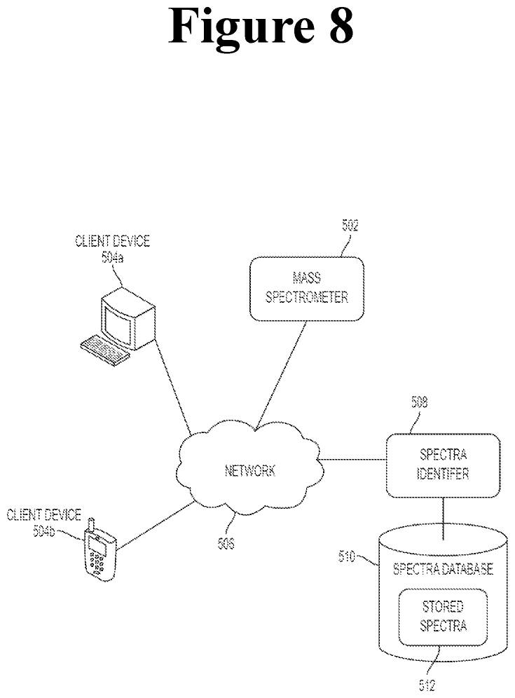

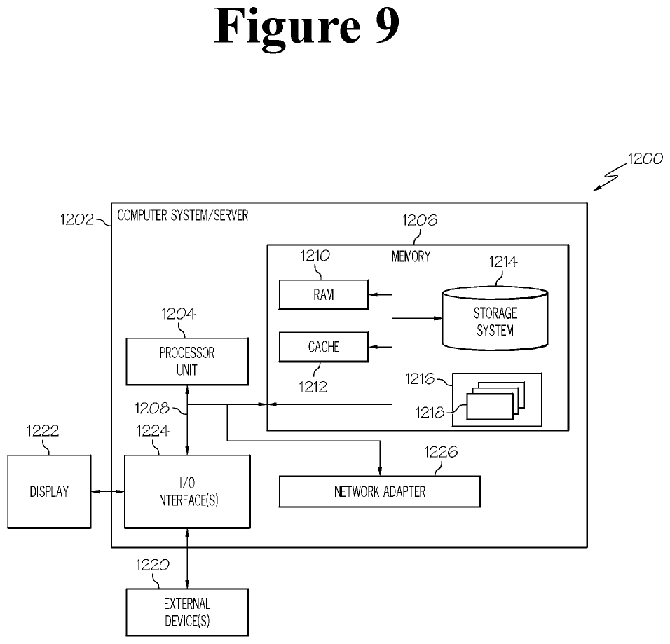

Example FIG. 8 illustrates a network configuration, in accordance with embodiments. Example FIG. 9 illustrates a cloud computing node, in accordance with embodiments.

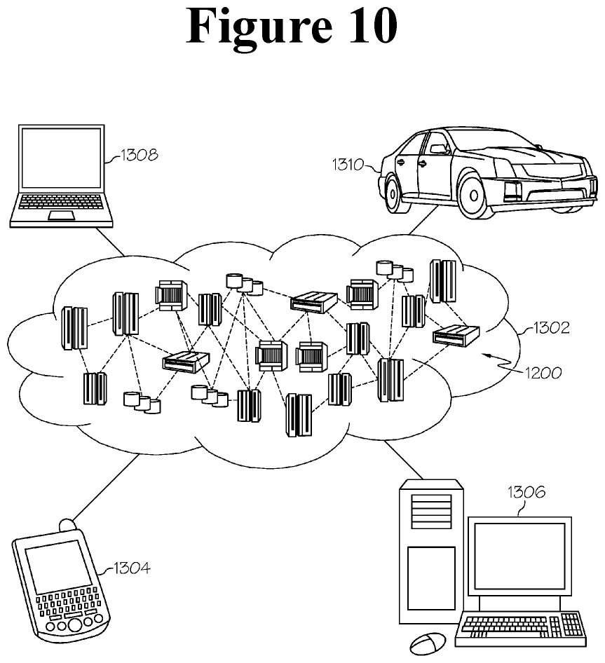

Example FIG. 10 illustrates a cloud computing environment, in accordance with embodiments.

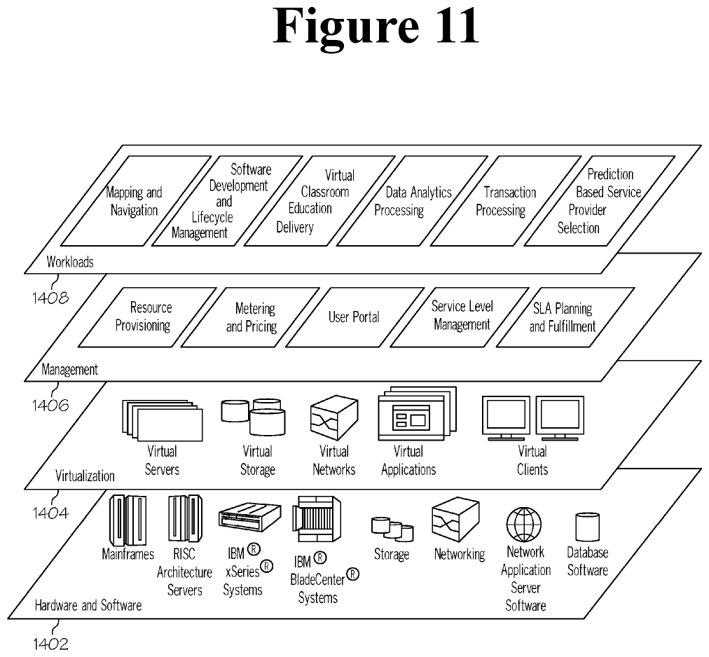

Example FIG. 11 illustrates abstraction model layers, in accordance with embodiments.

### DESCRIPTION

Example FIG. 1 shows the integrated disease diagnosis system, in accordance with embodiments. In embodiments, the generating of a mass spectrometry profile may use a mass spectrometer 302, such as a Matrix-Assisted Laser Desorption/Ionization Time-of-Flight Mass Spectrometer (MALDI-TOF MS) 302.

For example, in embodiments, an integrated disease diagnosis system may include a Matrix-Assisted Laser Desorption/Ionization Time-of-Flight Mass Spectrometer (MALDI-TOF MS) 302. Samples may undergo a combination of processes by selected modules. In the sample preparation system 301, a sample goes through a predefined and preprogrammed sequence depending on diagnosis or screening purposes in an automatic sample preparation unit 311. In embodiments, for glycan extraction, multiple processing modules may be selected, which as sample reception, protein denaturation, deglycosylation, protein removal, drying, centrifugation, solid phase extraction, and/or spotting. After sample preparation, the sample loader 312 loads the samples onto the plates 306 and are dried in a sample dryer 307.

The samples may then be provided to the MALDI-TOF MS unit 302 (i.e. mass spectrometer) having an ion flight chamber 321 and/or a high voltage vacuum generator 322, in accordance with embodiments. A processing unit 323 in the MALDI-TOF MS may identify the mass/charge and its corresponding intensity. For the disease diagnostic purpose, those acquired mass and intensity data may be reorganized to set up a standard mass list, in which a concept of the center of mass where intensities are balanced and equilibrated is introduced. A standard mass to charge list is defined based upon the machine accuracy and the center of mass concept. The stored spectrum data for each laser irradiation may also be used to set up the standard mass list.

In embodiments, diagnostic unit 303 may then compare, the spectra from a patient's sample with the pre-stored spectra and analyzes the pattern difference of the two spectra. The diagnostic unit 303 may then identify the presence and progress of the disease. In embodiments, as shown in example FIG. 1, diagnostic unit 303 may be internally integrated to the MALDI-TOF MS unit 302. In embodiments, diagnostic unit 303 may be either internal or external to a mass spectrometer system. In embodiments, a diagnostic unit may be cloud based. In embodiments, a diagnostic unit may be networked to a mass spectrometer system by a local network (e.g. an intranet network), a public network (e.g. the internet), or any other network as appreciated by those skilled in the art. In embodiments, a diagnostic unit may be coupled to an artificial intelligence engine and/or to one or more processors that implement deep learning algorithms.

In embodiments, the comparing and/or the diagnosing includes artificial intelligence, machine learning, and/or pattern matching.

Embodiments relate to a system for diagnosing infectious diseases. The system may include an extraction unit configured to extract at least one substance from a sample of a test subject. The system may include a mass spectrometer unit configured to generating a mass spectrometry profile from the at least one substance. The system may include a comparison unit configured to compare the generated mass spectrometry profile to a database including a plurality of predetermined mass spectrometry profiles associated with known infectious diseases. The system may include a diagnostic unit configured to diagnose the test subject with at least one of the known infectious diseases if the generated mass spectrometry profile is substantially similar to at least one of the predetermined mass spectrometry profiles associated with the at least one of the known infectious diseases.

Example FIGS. 2A and 2B show and example input spectrum 860 and corresponding graph 862 of peaks of input spectrum 860 generated by a mass spectrometer, in accordance with embodiments. Example FIG. 2B, illustrates an analytical result specifically identifies the three highest peaks, respectively peaks 864a, 864b, and 864c, in input spectrum 860 as displayed in peak graph 862.

Example FIG. 3 illustrates a process of diagnosing infectious diseases, in accordance with embodiments. The method may include step 2 of extracting at least one substance from a sample of a test subject. The method may include step 4 of generating a mass spectrometry profile from the at least one substance. The method may include step 6 of comparing the generated mass spectrometry profile to a database including a plurality of predetermined mass spectrometry profiles associated with known infectious diseases. The method may include step 8 of diagnosing the test subject with at least one of the known infectious diseases if the generated mass spectrometry profile is substantially similar to at least one of the predetermined mass spectrometry profiles associated with the at least one of the known infectious diseases.

Example FIG. 4 illustrates substance extraction 14 from a test subject 10, in accordance with embodiments. A test subject 10, such as a human being or an animal under diagnosis, may be subject to a sample collection 12 (e.g. swabbing of saliva, drawing blood, etc.). Once the sample is collected, the sample may be subject to substance extraction 14, where parts or aspects of the sample are separated for the purposes of diagnostics. In embodiments, the substance extraction 14 may be an automated substance extraction 17.

In embodiments, the substance may be a segment of an RNA gene sequence and/or a segment of a DNA gene sequence 18. In embodiments, the substance may be a protein, a glycoprotein, a nucleocapsid protein, an envelop protein, and/or a spike protein 20. In embodiments, the substance may be a glycan, a N-linked glycan, and/or an O-linked glycan 22.

Example FIG. 5 illustrates swabbing extraction of nose, throat, and/or saliva from a test subject, in accordance with embodiments. In embodiments, the extracting the substance from the sample of the test subject may include swabbing the sample from the test subject nose, throat, and/or saliva using a swab 24. The substance may be extracted from the swab 14.

Example FIG. 6 illustrates blood extraction from a test subject, in accordance with embodiments. In embodiments, the extracting the substance from the sample of the test subject includes drawing the sample in the form of blood 24 from the test subject and extracting the substance from the blood 14. In embodiments, the extracting the substance from the blood 14 includes extracting serum from the blood 28 and extracting the at least one substance from the serum 30. In embodiments, the extracting the substance from the blood includes an automated process.

Example FIG. 7 illustrates diagnosis of a test subject, in accordance with embodiments. In embodiments, diagnosing 8 a test subject 10 includes identification of known infectious diseases 30 by analyzing the generated mass spectrometry profile. In embodiments, the diagnosing 8 the test subject 10 with the at least one of the known infectious diseases includes analyzing the generated mass spectrometry profile to determine a status of the at least one of the known infectious diseases 32.

In embodiments, the status of the at least one of the known infectious disease 32 includes identification of at least one pathogen virus, identification of an antigen, and/or identification of the immunity status 34 of the test subject 10 to the at least one pathogen virus. In embodiments, the identification of the at least one pathogen virus includes identification of at least one viral substance. In embodiments, the identification of the immunity status of the test subject includes the identification of at least one antibody of the at least one pathogen virus.

Example FIG. 8 shows spectra identifier 508 configured to communicate, via network 506, with mass spectrometer 502 and client devices 504a, 504b. Network 506 may correspond to a LAN, a wide area network (WAN), a corporate intranet, the public Internet, or any other type of network configured to provide a communications path between networked computing devices. The network 506 may also correspond to a combination of one or more LANs, WANs, corporate intranets, and/or the public Internet.

Although FIG. 8 only shows two client devices, distributed application architectures may serve tens, hundreds, or thousands of client devices. Moreover, client devices 504a and 504b (or any additional client devices) may be any sort of computing device, such as an ordinary laptop computer, desktop computer, network terminal, wireless communication device (e.g., a cell phone or smart phone), and so on. In some embodiments, client devices 504a and 504b can be dedicated to mass spectrometry and/or bacteriological research. In other embodiments, client devices 504a and 504b may be used as general purpose computers that are configured to perform a number of tasks and need not be dedicated to mass spectrometry or bacteriological research. In still other embodiments, the functionality of spectra identifier 508 and/or spectra database 510 can be incorporated in a client device, such as client devices 504a and/or 504b. In even other embodiments, the functionality of spectra identifier 508 and/or spectra database 510 can be incorporated into mass spectrometer 502.

Mass spectrometer 502 can be configured to receive an input material e.g., LA and/or LTA, and generate one or more spectra as output. For example, mass spectrometer 502 can be an electrospray ionization (ESI) tandem mass spectrometer or a SAWN-based mass spectrometer. In some embodiments, the output spectra can be provided to another device; e.g., spectra identifier 508 and/or spectra database 510, perhaps to be used as an input to the device. In other embodiments, the output spectra can be displayed on mass spectrometer 502, client devices 504a and/or 504b, and/or spectra identifier 508.

Spectra identifier 508 can be configured to receive, as an input, one or more spectra from mass spectrometer 502 and/or client device(s) 504a and/or 504b via network 506. In some embodiments, spectra identifier can be configured to directly receive input spectra via keystroke, touchpad or similar data input to spectra identifier 508, hard-wired connection(s) to mass spectrometer 502 and/or client device(s) 504a and/or 504b, accessing storage media configured to store input spectra (e.g., spectra database 510, flash media, compact disc, floppy disk, magnetic tape), and/or any other technique to directly provide input spectra to spectra identifier 508.

Spectra identifier 508 may be configured to generate results of spectra identification by comparing one or more input spectra to stored spectra 512. For example, stored spectra 512 can be known precursor ion mass spectrometry spectra. As shown in example FIG. 8, stored spectra 512 can reside in spectra database 510. When performing spectra identification, spectra identifier 508 can access and/or query spectra database 510 to retrieve part or all of stored spectra 512. In some embodiments, spectra identifier 508 can perform the comparison task directly; while in other embodiments, part or all of the spectra identification task can be performed by spectra database 510, perhaps by executing one or more query language commands upon stored spectra 512.

While FIG. 8 shows spectra identifier 508 and spectra database 510 directly connected, in other embodiments, spectra identifier 508 can include the functionality of spectra database 510, including storing stored spectra 512. In still other embodiments, spectra identifier 508 and spectra database 510 can be connected via network 506.

Upon identifying the input spectra, spectra identifier 508 can be configured to provide content at least related to results of spectra identification, as requested by client devices 504a and/or 504b. The content related to results of spectra identification can include, but is not limited to, web pages, hypertext, scripts, binary data such as compiled software, images, audio, and/or video. The content can include compressed and/or uncompressed content. The content can be encrypted and/or unencrypted. Other types of content are possible as well.

In embodiments, the database 510 is stored in a cloud computing environment. In embodiments, the comparing the generated mass spectrometry profile to the database includes transmitting the generated mass spectrometry profile to the cloud computing environment.

In embodiments, the generating the mass spectrometry profile is performed in one of a plurality of mass spectrometers 502 and the plurality of mass spectrometers 502 are networked 506 to the cloud computing environment. In embodiments, the database 510 stored in the cloud computing environment is configured to process geographic attributes of the known infectious diseases. For infectious diseases, geographic propagation of the disease is an important factor in curbing the spread of the disease. Since embodiments relate to an electronic diagnosis of infectious diseases in a server environment (e.g. a cloud computing environment), geographic attributes can be appreciated and used for both the diagnosis and the control of the spread of the infectious disease. In embodiments, at least one component of the cloud computing environment is configured to monitor the known infectious diseases for epidemiological surveillance. In embodiments, at least one component of the cloud computing environment is configured to send information to authorities of the geographic attributes of the known infection diseases. In embodiments, the cloud computing environment is configured to update a local database proximate to at least one of the plurality of mass spectrometers.

Example FIG. 9 illustrates a schematic of an example of a cloud computing node, in accordance with embodiments. Cloud computing node 1200 is only one example of a suitable cloud computing node and is not intended to suggest any limitation as to the scope of use or functionality of embodiments described herein. Regardless, cloud computing node 1200 is capable of being implemented and/or performing any of the functionality set forth hereinabove. The term processing node is a logical concept. Any number of central processing units with any number of cores or machines can be in a single processing node.

In cloud computing node 1200 there is a computer system/server 1202, which is operational with numerous other general purpose or special purpose computing system environments or configurations. Examples of well-known computing systems, environments, and/or configurations that may be suitable for use with computer system/server 1202 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 1202 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 1202 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 9, computer system/server 1202 in cloud computing node 1200 is shown in the form of a general-purpose computing device. The components of computer system/server 1202 may include, but are not limited to, one or more processors or processing units 1204, a system memory 1206, and a bus 1208 that couples various system components including system memory 1206 to processor 1204.

Bus 1208 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 1202 typically includes a variety of computer system readable media. Such media may be any available media that is accessible by computer system/server 1202, and it includes both volatile and non-volatile media, removable and non-removable media.

The system memory 1206 can include computer system readable media in the form of volatile memory, such as random access memory (RAM) 1210 and/or cache memory 1212. Computer system/server 1202 may further include other removable/non-removable, volatile/non-volatile computer system storage media. By way of example only, storage system 1214 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1208 by one or more data media interfaces. As will be further depicted and described below, memory 1206 may include at least one program product having a set (e.g., at least one) of program modules that are configured to carry out the functions of various embodiments of the invention.

Program/utility 1216, having a set (at least one) of program modules 1218, may be stored in memory 1206 by way of example, and not limitation, as well as an operating system, one or more application programs, other program modules, and program data. Each of the operating system, one or more application programs, other program modules, and program data or some combination thereof, may include an implementation of a networking environment. Program modules 1218 generally carry out the functions and/or methodologies of various embodiments of the invention as described herein.

Computer system/server 1202 may also communicate with one or more external devices 1020 such as a keyboard, a pointing device, a display 1222, etc.; one or more devices that enable a user to interact with computer system/server 1202; and/or any devices (e.g., network card, modem, etc.) that enable computer system/server 1202 to communicate with one or more other computing devices. Such communication can occur via I/O interfaces 1224. Still yet, computer system/server 1202 can communicate with one or more networks such as a local area network (LAN), a general wide area network (WAN), and/or a public network (e.g., the Internet) via network adapter 1226. As depicted, network adapter 1226 communicates with the other components of computer system/server 1202 via bus 1208. It should be understood that although not shown, other hardware and/or software components could be used in conjunction with computer system/server 1202. Examples, include, but are not limited to: microcode, device drivers, redundant processing units, external disk drive arrays, RAID systems, tape drives, and data archival storage systems, etc.

Example FIG. 10 illustrates cloud computing environment 1302, in accordance with embodiments. As shown, cloud computing environment 1302 comprises one or more cloud computing nodes 1200 with which local computing devices used by cloud consumers, such as, for example, personal digital assistant (PDA) or cellular telephone 1304, desktop computer 1306, laptop computer 1308, and/or automobile computer system 1310 may communicate. Nodes 1200 may communicate with one another. They may be grouped (not shown) physically or virtually, in one or more networks, such as Private, Community, Public, or Hybrid clouds as described hereinabove, or a combination thereof. This allows cloud computing environment 1302 to offer infrastructure, platforms and/or software as services for which a cloud consumer does not need to maintain resources on a local computing device. It is understood that the types of computing devices 1304, 1306, 1308, 1310 shown in FIG. 10 are intended to be illustrative only and that computing nodes 1200 and cloud computing environment 1302 can communicate with any type of computerized device over any type of network and/or network addressable connection (e.g., using a web browser).

Example FIG. 11 illustrates a set of functional abstraction layers provided by cloud computing environment 1302 (FIG. 10), in accordance with embodiments. It should be understood in advance that the components, layers, and functions shown in FIG. 11 are intended to be illustrative only and embodiments are not limited thereto. As depicted, the following layers and corresponding functions are provided:

Hardware and software layer 1402 includes hardware and software components. Examples of hardware components include mainframes; RISC (Reduced Instruction Set Computer) architecture based servers; storage devices; networks and networking components. Examples of software components include network application server software and database software.

Virtualization layer 1404 provides an abstraction layer from which the following examples of virtual entities may be provided: virtual servers; virtual storage; virtual networks, including virtual private networks; virtual applications and operating systems; and virtual clients. In one example, management layer 1406 may provide the functions of processing unit 68. Workloads layer 1408 provides examples of functionality for which the cloud computing environment may be utilized.

Aspects of the present invention have been discussed above with reference to flowchart illustrations and/or block diagrams of methods, apparatus (systems) and computer program products according to various embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It will be obvious and apparent to those skilled in the art that various modifications and variations can be made in the embodiments disclosed. This, it is intended that the disclosed embodiments cover the obvious and apparent modifications and variations, provided that they are within the scope of the appended claims and their equivalents.

What is claimed is:

1. A method comprising:
extracting at least one substance from a sample of a test subject;
generating a mass spectrometry profile from the at least one substance;
comparing the generated mass spectrometry profile to a database comprising a plurality of predetermined mass spectrometry profiles associated with known infectious diseases; and
diagnosing the test subject with at least one of the known infectious diseases if the generated mass spectrometry profile is substantially similar to at least one of the predetermined mass spectrometry profiles associated with the at least one of the known infectious diseases.

2. The method of claim 1, wherein the at least one substance comprises a segment of an RNA gene sequence and/or a segment of a DNA gene sequence.

3. The method of claim 1, wherein the substance is at least one of:
a protein;
a glycoprotein;
a nucleocapsid protein;
an envelop protein; and/or
a spike protein.

4. The method of claim 1, wherein the substance comprises at least one of:
a glycan;
a N-linked glycan; and/or
an O-linked glycan.

5. The method of claim 1, wherein the extracting the at least one substance from the sample of the test subject comprises:
swabbing the sample from the test subject nose, throat, and/or saliva using a swab; and
extracting the at least one substance from the swab.

6. The method of claim 1, wherein the extracting the at least one substance from the sample of the test subject comprises:
drawing the sample in the form of blood from the test subject; and
extracting the at least one substance from the blood.

7. The method of claim 6, wherein the extracting the at least one substance from the blood comprises:
extracting serum from the blood; and
extracting the at least one substance from the serum.

8. The method of claim 7, wherein the extracting the at least one substance from the blood comprises an automated process.

9. The method of claim 1, wherein the generating the mass spectrometry profile comprises using a Matrix-Assisted Laser Desorption/Ionization Time-of-Flight Mass Spectrometer (MALDI-TOF MS).

10. The method of claim 1, wherein:
the database is stored in a cloud computing environment; and
the comparing the generated mass spectrometry profile to the database comprising transmitting the generated mass spectrometry profile to the cloud computing environment.

11. The method of claim 10, wherein the database stored in the cloud computing environment is configured to process geographic attributes of the known infectious diseases.

12. The method of claim 11, wherein:
the generating the mass spectrometry profile is performed in one of a plurality of mass spectrometers;
the plurality of mass spectrometers are networked to the cloud computing environment; and
at least one component of the cloud computing environment is configured to monitor the known infectious diseases for epidemiological surveillance.

13. The method of claim 12, wherein at least one component of the cloud computing environment is configured to send information to authorities of the geographic attributes of the known infection diseases.

14. The method of claim 12, wherein the cloud computing environment is configured to update a local database proximate to at least one of the plurality of mass spectrometers.

15. The method of claim 1, wherein the diagnosing the test subject with the at least one of the known infectious diseases comprises analyzing the generated mass spectrometry profile to determine a status of the at least one of the known infectious diseases.

16. The method of claim 15, wherein the status of the at least one of the known infectious diseases comprises at least one of:
identification of at least one pathogen virus;
identification of an antigen; and/or
identification of the immunity status of the test subject to the at least one pathogen virus.

17. The method of claim 16, wherein the identification of the at least one pathogen virus comprises identification of at least one viral substance.

18. The method of claim 16, wherein the identification of the immunity status of the test subject comprises identification of at least one antibody of the at least one pathogen virus.

19. The method of claim 1, wherein at least one of the comparing and/or the diagnosing comprises at least one of:
artificial intelligence;
machine learning; and/or
pattern matching.

20. A system comprising:
an extraction unit configured to extract at least one substance from a sample of a test subject;
a mass spectrometer unit configured to generating a mass spectrometry profile from the at least one substance;
a comparison unit configured to compare the generated mass spectrometry profile to a database comprising a plurality of predetermined mass spectrometry profiles associated with known infectious diseases; and
a diagnostic unit configured to diagnose the test subject with at least one of the known infectious diseases if the generated mass spectrometry profile is substantially similar to at least one of the predetermined mass spectrometry profiles associated with the at least one of the known infectious diseases.
